(19) United States
(12) Patent Application Publication
(10) Pub. No.: US 2016/0262403 A1
(43) Pub. Date: Sep. 15, 2016

(54) CUSHIONING FOAMS CONTAINING AROMATIC WOOD PARTICLES

(71) Applicant: PETERSON CHEMICAL TECHNOLOGY LLC, West Lake Hills, TX (US)

(72) Inventors: BRUCE W. PETERSON, Fort Smith, AR (US); MARK L. CRAWFORD, Rudy, AR (US)

(73) Assignee: PETERSON CHEMICAL TECHNOLOGY LLC, West Lake Hills (US)

(21) Appl. No.: 15/036,320

(22) PCT Filed: Nov. 25, 2013
(86) PCT No.: PCT/US2013/071721

§ 371 Date: May 12, 2016

(51) Int. Cl. A01N 65/06; A01N 25/16; A61L 9/013; A01K 1/035; A47C 27/14; A47C 31/00; A01K 1/015; C08J 9/00; C11B 9/00

(52) U.S. Cl. CPC A01N 65/06; C08J 9/0061; A01N 25/16; A61L 9/013; C11B 9/00; A47C 27/14; A47C 31/005; A47C 31/007; A01K 1/0157; A01K 1/0353; C08J 2205/06; C08J 2375/04; C08J 2207/00; C08J 2497/02; C08J 2361/28; C08J 2321/02

(57) ABSTRACT

Methods and combinations for making and using one or more cellular foam layers comprising flexible cellular foam and wood particles having aromatic properties, where said cellular foam layers may be located on, under, or in cushioning foams and mattresses, or placed between on, under, within, or between other layering substrates. The foam containing aromatic wood particles may be used in mattresses, pillows, bedding products, medical cushioning foams, pet beds, outdoor bedding pads, outdoor pillows, and other cushioning products.

### TECHNICAL FIELD

This invention relates to methods for making and using one or more foam layers comprising flexible cellular foam and wood particles having aromatic properties, and said layers are located on, under, or in mattresses, bedding, and cushioning products. This invention more specifically relates to various types of foams containing wood particles having aromatic properties including, but not necessarily limited to, mattresses, pillows, mattress topper pads, quilted toppers, medical mattresses, pet beds, outdoor bedding pads, outdoor pillows, and other cushioning products.

### TECHNICAL BACKGROUND

Cedar shavings have been used in pet beds to act as a pest or odor repellant. Cedar oil has been tested and proven effective in repelling insects such as fleas, ticks, mosquitos, black flies, and in reducing animal odor. EPA profiles the use of cedarwood oil as a natural repellant/feeding depressant and fungicide used in houses and on pets or their bedding to repel fleas, moths, and mildew. Cedar wood encompasses a variety of species, including, but not necessarily limited to, Spanish Cedar, Eastern Cedar, Juniper, and aromatic Red Cedar. Cedarwood oil, or cedar oil, is widely used as a fragrance in soaps, air fresheners, household detergents, and cosmetics.

U.S. Pat. No. 5,320,006 disclosed a mattress or bed for pets with stuffing material treated with white cedar oil. The stuffing material comprises cedar shavings. White cedar shavings are mixed with an olive or mineral oil containing white cedar oil.

U.S. Pat. No. 4,226,944 disclosed fragrance-emitting articles comprising polyurethane foam containing a particulate filler and a fragrance material, and a method of making such articles. The method includes pre-mixing particulate filler and fragrance and dispersing the pre-mix in a liquid polyol, and using the premix to produce polyurethane foam having a controlled rate of release of the fragrance by desorption and diffusion. Preferred particulates are treated and untreated clays, ground limestone, precipitated calcium carbonate, alumina, aluminum silicate, barites, calcium silicate, silica, zirconia, titanium dioxide, soap, and synthetic detergents in solid form. Polyurethane foams containing particulate filler and fragrance material are suitable for use as pomanders, garment sachets, room air-fresheners, and the like.

U.S. Pat. No. 4,618,629 disclosed polyurethane foams having a thermoplastic or thermosetting particulate resin carrying a fragrance incorporated during formation of the polyurethane foam. The fragrance is released over an extended period of time. Preferred particulates, which are compatible with both the fragrance and foam-forming formulation, are acrylate resins such as polymethylmethacrylate, polyethylacrylate, polyhydroxyethylacrylate, and copolymers thereof.

It is useful and desirable to develop improved comfort layers that have aromatic wood particles in a cushion or mattress.

### SUMMARY

There is provided, in one non-limiting form, methods of forming flexible cellular foam with aromatic wood particles (referred hereafter as “AW Foam”) comprised of a flexible polyurethane foam and/or polyester polyurethane foam, which may be open or partially open celled in nature, and a plurality of wood particles dispersed therein having aromatic properties. Other performance modifying additives may optionally be incorporated into the foam. The AW Foam may contain aromatic wood particles in the range of about 0.1% independently to about 50% on a weight basis. Optionally, the flexible cellular foam may be synthetic or natural latex foam or melamine foam.

There is also provided, in one non-limiting form, methods of increasing the aromatic fragrance of the AW Foam by pre-treating the wood particles with an extracted essential oil. Optionally, the aromatic fragrance may be added to a plasticized or un-plasticized tri-block copolymer polymer and used to control release an essential oil fragrance.

The AW Foam may be cut or molded in many structures such as, but not limited to, planar layers, convoluted layers, surface modified layers, 3D surface texturing, molded pillows, smooth molded surfaces, molded surfaces with regular or irregular patterns, or modified in any way as to generate a desired physical structure such as but not limited to hole punching, channeling, reticulation or other method known to the art of foaming for modifying the structure of foam. The AW Foam may be adhered in the cushion or mattress composite with adhesive or melting of a thermoplastic on the foam surface and allowing the thermoplastic to re-solidify and lock the foam in place on the substrate foam.

There is also provided, in a non-restrictive embodiment, combinations of suitable layering substrates including, but not limiting to, flexible polyurethane foam, latex foam, flexible melamine foam, and other substrates (such as fibers in woven or non-woven form) with one or more AW Foams. Articles that may be manufactured from these combinations include, but are not necessarily limited to, mattresses, mattress toppers, pillows, bedding products, pet beds, quilted mattress toppers, pillow or mattress inserts, contoured support foam, outdoor bedding pads, outdoor bedding pillows, or other cushioning products.

### BRIEF DESCRIPTION OF THE DRAWINGS

FIG. 1 is an example blending system of polyol and AW particles;

FIG. 2 is a first example of construction using a cushion and/or mattress application implementing the AW foams described herein;

FIG. 3 is the second example construction using a cushion and/or mattress application implementing the AW foams described herein;

FIG. 4 is the third example construction using a cushion and/or mattress application implementing the AW foams described herein;

FIG. 5 is the fourth example construction using a cushion and/or mattress application implementing the AW foams described herein;

FIG. 6 is the fifth example construction using a cushion and/or mattress application implementing the AW foams described herein;

FIG. 7 is the sixth example construction using a cushion and/or mattress application implementing the AW foams described herein;

FIG. 8 is the seventh example construction using a cushion and/or mattress application implementing the AW foams described herein;

FIG. 9 is the eighth example construction using a cushion and/or mattress application implementing the AW foams described herein;

FIG. 10 is the ninth example construction using a cushion and/or mattress application implementing the AW foams described herein;

FIG. 11 shows an example breakdown of lateral mattress zones in a cushion and/or mattress application;

FIG. 12 shows an example breakdown of longitudinal mattress zones in a cushion and/or mattress application;

FIG. 13 is an example of a molded pillow product where the entire structure is molded from AW Foam;

FIG. 14 is an example of a molded pillow product where the AW Foam is a region or layer within the pillow;

FIG. 15 is a photograph of aromatic wood particles in open cell flexible polyurethane foam; and

FIG. 16 is a close-up photograph of viscoelastic polyurethane foam made with 10 wt % cedar powder added.

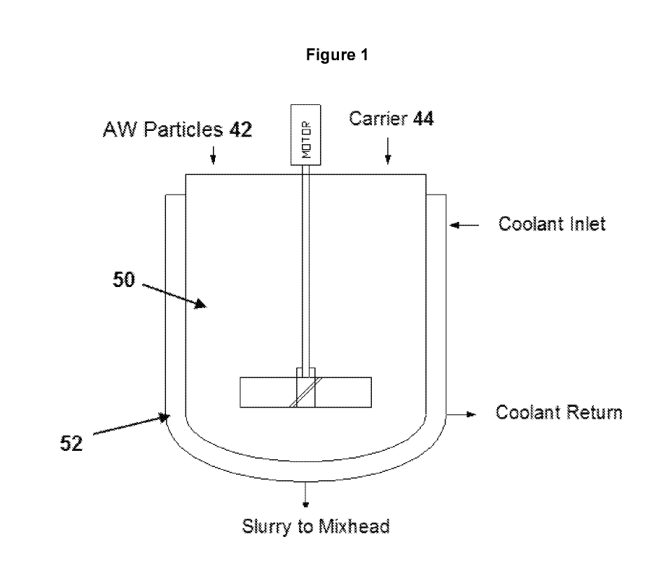

It will be appreciated that FIGS. 1-14 are schematic and that the various elements are not necessarily to scale or proportion, and that many details have been removed or simplified for clarity, and thus the methods and compositions are not necessarily limited to the embodiments depicted in the Figures.

Before the methods and compositions are explained in detail, it is to be understood that these methods and compositions are not limited in its application to the details of construction and the arrangements of the components set forth in the following description or illustrated in drawings. Also, it is understood that the phraseology and terminology used herein are for the purpose of description and should not be regarded as limiting.

### DETAILED DESCRIPTION

It is useful to develop improved comfort layers that have aromatic wood particulates in a cushion or mattress by incorporating one or more AW Foam layers comprising a flexible cellular foam and aromatic wood particles, and said one or more AW Foam layers are used on, under, or within mattresses, pillows, bedding products, medical cushioning foams, outdoor bedding pads, pet beds, outdoor pillows, and other cushioning products.

Flexible cellular foams may include, but are not limited to, open cell polyurethane foam, partially open cell polyurethane foam, open cell polyester polyurethane foam, partially open cell polyester polyurethane foam, latex foam, melamine foam, and combinations thereof.

AW Foams are comprised of an open or partially open celled flexible polyurethane or polyester foam that has one or more aromatic wood materials (such as in the form of particulates) randomly dispersed throughout the foam. Alternatively, the wood material particulates may be uniformly dispersed throughout the foam.

The AW Foam contains wood material in the range of about 0.1% independently to about 50% on a weight basis in the foam. Alternatively, the AW Foam contains wood material in the range of about 1% independently to about 40% on a weight basis in the foam, and in another non-limiting embodiment in the range of about 2% independently to about 30% on a weight basis in the foam, and, in a different non-restrictive version, in the range of about 2% independently to about 25% on a weight basis in the foam. The term “independently” as used in association with various ranges herein means that any lower threshold may be combined with any upper ratio to form a suitable alternative range.

Aromatic wood particles or particulates may include, but are not necessarily limited to, wood particles of Acacia, African Mahogany, Algum, Almond, Almug, Aloes, Apple, Apricot, Ash, Balm, Balsam, Bdellium, Black Locust, Bloodwood, Bocote, Boxthorn, Boxwood, Bramble, Brier, Broom, Bubinga, Butternut, Cambara, Camphire, Canarywood, Carob, Cassia, Castor, Catalpa, Cedar, Chechen, Cherry, Chestnut, Cinnamon, Cocobolo, Colorwood, Cypress, Dakota burl, Dymondwood, Eastern red cedar, Ebony, Elm, Eucalyptus, Fig, Fir, Frankincense, Galbanum, Goncalo Alves, Gopher, Green Bay, Gum, Hazel, Heath, Holm, Hyssop, Jarrah, Jatoba, Juniper, Kingwood, Koa, Lacewood, Locust, Lotus, Mallows, Maple, Mesquite, Mopane, Mulberry, Mustard, Myrrh, Myrtle, Oak, Oil, Oleander, Olive, Osage Orange, Pacific Yew, Padauk, Palm, Papyrus, Pau Amarillo, Pau Rosa, Persimmon, Pick Ivory, Pine, Pistachio, Plane, Pomegranate, Poplar, Purpleheart, Red Mulberry, Red Oak, Rose, Rosewood, Senna, Spice, Shittim, Storax, Sycamine, Sycamore, Tamarisk, Teil, Terebinth, Thorn, Thyine, Tulipwood, Vine, Walnut, Wenge, White Oak, Willow, Wormword, Zebrawood, and combinations thereof. It is expected that wood particulates made from all of these woods will be aromatic by their nature to some extent.

Suitable aromatic wood particles may include, but are not necessarily limited to, wood particles of Spanish Cedar, Eastern Red Cedar, Juniper, aromatic Red Cedar, White Cedar, Eucalyptus, Cypress, Western Red Cedar, and combinations thereof.

The aromatic wood material may include, but are not necessarily limited to, shavings, flake, powder, spherical, splinters, or other various particulate forms. A suitable size of aromatic wood materials may be between about 0.1 microns independently to about 3000 microns, alternatively between about 1 micron independently to about 2000 microns, and in another non-limiting embodiment between about 10 microns independently to about 1000 microns.

Aromatic wood particles may be obtained by many different size reduction and comminution techniques. A non-limiting list of size reduction processes include jaw crushers, gyratory crushers, roll crushers, impact breakers, pan crushers, tumbling mills, non-rotary ball mill, particle-size classifiers used with grinding mills, hammer mills, ring-roller mills, disk attrition mills, jet mills, cutting mills, saw mill, sanding disks, high speed solid impingements such as sand-blasting, and combinations thereof.

In a non-limiting embodiment, aromatic wood particles may be sized by air classification, static screens, rotary sifter, centrifugal screen, vibratory screen, gyratory screen, cyclone separator, and combinations thereof. Alternatively, the particles may be sized based on wet classification techniques such a cone type classifier, liquid cyclone, hydroseparator, solid-bowl centrifuge, countercurrent classifier, jet sizer, supersorter, and combinations thereof.

In a non-limiting embodiment, aromatic wood particles may be dried before or after sizing to the suitable or required particulate size. The moisture content of the aromatic wood particles can be between 0.1 independently to 50% by weight of water in the wood. A preferred embodiment of this invention is to use kiln-dried wood, which has between 6 to 20% by weight of water in the wood. The aromatic wood may be additionally dried by a non-limiting list consisting of vacuum-shelf dryers, rotary dryers, vacuum rotary dryers, turbo-tray dryers, hearth furnaces, direct heat tray dryers, vibrating conveyor dryers, fluidizing bed dryers, cyclone separator dryers, and combinations thereof. If the AW particles are not dried, the moisture content can be determined through gravimetric methods by using a heated oven at about 110° C. and measuring the weight loss. Once the weight loss is determined, the water addition in the polyurethane foam formulation can be corrected to control the final foam density.

The AW Foam may also contain useful amounts of conventionally employed additives (“property-enhancing additives”) such as plasticized triblock copolymer gels, stabilizers, antioxidants, antistatic agents, antimicrobial agents, ultraviolet stabilizers, phase change materials, surface tension modifiers such as silicone surfactants, emulsifying agents, and/or other surfactants, solid flame retardants, liquid flame retardants, grafting polyols, compatible hydroxyl-containing chemicals which are completely saturated or unsaturated in one or more sites, solid or liquid fillers, anti-blocking agents, colorants such as inorganic pigments, carbon black, organic colorants or dyes, reactive organic colorants or dyes, heat-responsive colorants, heat-responsive pigments, heat-responsive dyes, pH-responsive colorants, pH-responsive pigments, pH-responsive dyes, fragrances, viscosity-modifiers such as fumed silica and clays, thermally conductive-enhancing additives such as aluminum and graphite, and combinations thereof, and other polymers in minor amounts and the like to an extent not affecting or substantially decreasing the desired properties of the AW Foam.

Aromatic essential oils and fragrances may include, but are not necessarily limited to, Agar oil, Ajwain oil, Angelica root oil, Anise oil, Asafoetida, Balsam oil, Basil oil, Bay oil, Bergamot oil, Black Pepper, Birch, Camphor, Cannabis flower, Caraway oil, Cardamom seed oil, Carrot seed oil, Cedarwood oil, Chamomile oil, Calamus Root, Cinnamon oil, Citronella oil, Clary Sage, Clove leaf oil, Coffee, Coriander, Costmary oil, Costus Root, Cranberry seed oil, Cubeb, Cumin oil/Black seed oil, Cypress oil, Cypriol, Curry leaf, Davana oil, Dill oil, Elecampane, Eucalyptus oil, Fennel seed oil, Fenugreek oil, Fir, Frankincense oil, Galangal, Galbanum, Geranium oil, Ginger oil, Goldenrod, Grapefruit oil, Henna oil, Helichrysum, Hickory nut oil, Horseradish oil, Hyssop, Idaho Tansy, Jasmine oil, Juniper berry oil, Laurus nobilis, Lavender oil, Ledum, Lemon oil, Lemongrass oil, Lime oil, Litsea cubeba oil, Mandarin, Marjoram, Melaleuca, Melissa oil, Mentha arvensis oil, Mint oil, Mountain Savory, Mustard oil, Myrrh oil, Myrtle, Neem oil, Neroli, Nutmeg, Orange oil, Oregano oil, Orris oil, Palo Santo, Parsley oil, Patchouli oil, Perilla, Peppermint oil, Petitgrain, Pine oil, Ravensara, Red Cedar oil , Roman Chamomile, Rose oil, Rosehip oil, Rosemary oil, Rosewood oil, Sage oil, Star anise oil, Sandalwood oil, Sassafras oil, Savory oil, Schisandra oil, Spearmint oil, Spikenard, Spruce, Star anise oil, Tangerine, Tarragon oil, Tea tree oil, Thyme oil, Tsuga, Turmeric, Valerian, Vetiver oil, Western red cedar oil, Wintergreen oil, Yarrow oil, Ylang-ylang, Zedoary, and combinations thereof.

Suitable aromatic essential oils and fragrances may include, but are not necessarily limited to, Cedarwood oil, Citronella oil, Cypress oil, Eucalyptus oil, Juniper berry oil, Lavender oil, Lemon oil, Lemongrass oil, Lime oil, Mint oil, Peppermint oil, Pine oil, Red Cedar oil, Western red cedar oil, Wintergreen oil, and combinations thereof.

The AW foam may have natural insect repellant properties based on addition of an adequate amount of one or more aromatic wood types, one or more essential oils, or combinations thereof to repel insects. Alternatively, the wood particulates have no aromatic odor in nature, but the aromatic fragrance is obtained by addition of one or more essential oils.

In a non-limiting embodiment, the aromatic wood particles' fragrance intensity may be increased by soaking, spraying, dripping, and other means of at least partially coating the wood particles prior to making flexible cellular foam. Alternatively or in addition to coating the aromatic wood particulates, the aromatic wood particulates may absorb or adsorb one or more aromatic essential oil and/or fragrance. Penetration of the essential oil in the wood particles may be helped by increasing temperature, increasing pressures, creating vacuum, using a solvent, and combinations thereof. Suitable solvents are, but not necessarily limited to, acetone, hexane, toluene, xylene, methyl formate, methyl acetate, and other solvents with boiling points less than 300° F.

Triblock copolymers may also be used to enhance or change aromatic characteristics without having to treat the wood particles. Essential oils may be blended in with a plasticizer and mixed with triblock copolymer resin to give an aromatic gelatinous triblock copolymer. Optionally, the essential oil and triblock copolymer may be combined without any plasticizer.

Triblock copolymers include, but are not necessarily limited to, (SB)n styrene-butadiene, (SEB)n, (SIS) styrene-isoprene-styrene block copolymers, (SEBS) styrene-ethylene-butylene-styrene block copolymers, (SEP) styrene-ethylene-propylene block copolymers, (SEEPS) styrene-ethylene-ethylene-propylene-styrene block copolymers, (SBS) styrene-butadiene-styrene block copolymers and the like. The term “n” here and elsewhere refers to the number of repeating polymer units. The triblock copolymers employed may have the more general configuration of A-B-A. The A component represents a crystalline polymer end block segment of polystyrene; and the B component represents an elastomeric polymer center block segment. These “A” and “B” designations are only intended to reflect conventional block segment designations.

Plasticizers suitable for plasticizing triblock copolymer resins are well known in the art. They include, but are not necessarily limited to, rubber processing oils such as paraffinic and naphthenic petroleum oils, highly refined aromatic-free paraffinic and naphthenic food and technical grade white petroleum mineral oils, synthetic oils and natural oils and polyols made from natural oils and natural polyols. Synthetic oils include, but are not necessarily limited to, high viscosity oligomers such as non-olefins, isoparaffins, paraffins, aryl and/or alkyl phosphate esters, aryl and/or alkyl phosphite esters, polyols, and glycols.

One method of producing a gelatinous triblock copolymer with a Western Red Cedar odor is to mix a plasticizer with Western Red Cedar essential oil in a ratio of 99:1. The mixture is heated to about 180° F. and mixed with Kraton G1651 linear triblock copolymer to give gel particles with Western Red Cedar oil infused in the gel. The gel particles are then added to the wood particles to have a slow release fragrant blend which is contained within a flexible cellular foam.

AW Foams may be prepared by a method or methods including batch-wise or continuous pouring in a form, mold or on a bun production line, and in one non-limiting embodiment, the AW material may be incorporated or blended into the polyol blend in a batch-wise or continuous process in a blending system such as a continuous stirred tank, static mixing elements, air mixers, or any other equipment known in the skill of the art that is used for mixing solids and additives with liquids.

One non-limiting embodiment of adding AW particles 42 to the compatible carrier 44 is by adding the AW particles 42 into a compatible carrier in a mix tank 50, as schematically illustrated in FIG. 1. A typical mix tank 50 may have a heating/cooling jacket 52 for controlling the temperature within the tank. The carrier is added to the mixing tank and then the AW particles 42 are mixed into the carrier while agitating. While mixing, the AW particles 42 may be added to the tank gradually or all at once. Alternatively, the AW particles 42 may be added to the mixing tank first and then the compatible carrier added to the tank while mixing. Another non-limiting method of adding AW particles to the compatible carrier is by transferring the AW particles into a mix chamber using an auger, where the AW particles and compatible carrier are mixed prior to adding other chemicals required to make polyurethane foam. Alternatively, mixing may be performed directly into the main mix head or can be mixed in a separate mix head and the AW particles and compatible carrier mixture fed into the main mix head with the other formulation components. Another non-restrictive method may be to use the auger to mix the AW particles and compatible carrier together while augering to the main mix head. It will be appreciated that the method described herein is not limited to these examples, since there are many possible combinations for combining AW particles with a compatible carrier before incorporating AW particles into final polyurethane foam.

The AW Foam can be poured in a standard bun form on a conveyor, poured in a mold having planar or non-planar surfaces, textured with 2D and 3D modification, or poured in a mold with rods to make the foam perforated.

In one non-limiting embodiment, one or more AW Foams may be added within or on the surface or in any location within the interior cavity of a mold for making molded products such as, but not limited to, pillows, mattresses, mattress toppers, pet beds, seat cushions, and individual substrate components added to the mold to react, bind, or encapsulate the AW Foam.

It will be appreciated that the method described herein is not limited to these examples, since there are many possible combinations for making AW Foams with open or partially open cell polyurethane foams or polyester foams that can be used in cushion foams or mattresses. Further details about making foams, including gel-foams, and the foam and gel-foam compositions so made may be seen in U.S. Patent Application Publication Nos. ______ and ______ (U.S. Ser. Nos. 13/932,492 and 13/932,535, respectively), incorporated herein by reference in their entirety.

### Applications of the AW Foam

AW Foam can be manufactured and combined with substrate foams for use in a variety of bedding applications, including but not necessarily limited to, mattresses, pillows, pillow toppers, mattress toppers, quilted toppers, body support foam, pet beds, outdoor bedding pads, outdoor pillows, or other cushioning materials.

AW Foam may be used as a single component in an article, such as a pet bed where the cushioning component consists only of AW Foam. In one non-limiting embodiment, AW foam is contained within a fabric cover and used as a pet bed.

Layering substrates in combination with one or more AW Foams and optional property-enhancing materials described herein may find utility in a very wide variety of applications. Suitable layering substrates include, but are not limited to, flexible polyurethane foam, flexible polyester polyurethane foam, latex foam, flexible melamine foam, and other substrates (such as fibers in woven or non-woven form), and combinations thereof. More specifically, in other non-limiting embodiments, the combination of AW Foam and substrate would be suitable as pillows or pillow components, including, but not necessarily limited to, pillow wraps or shells, pillow cores, pillow toppers, for the production of medical comfort pads, medical mattresses and similar comfort and support products, and residential/consumer mattresses, mattress toppers, pet beds, outdoor bedding pads, outdoor pillows, and similar comfort and support products, typically produced with conventional flexible polyurethane foam or fiber. All of these uses and applications are defined herein as “bedding products” or cushioning products.

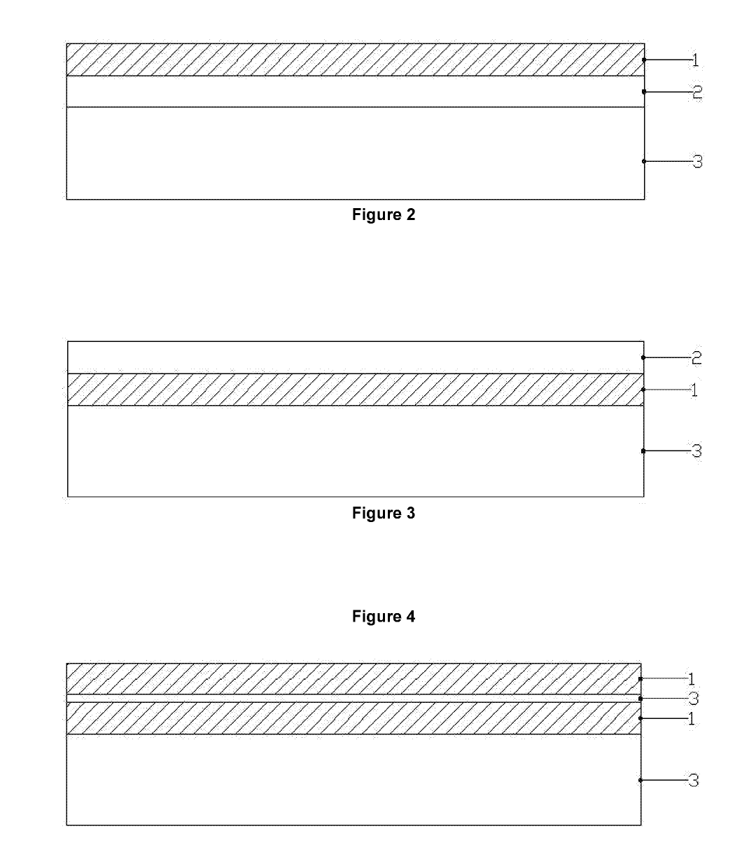

FIG. 2 is a first example of construction using a cushion and/or mattress application. The base of the section is a prime foam layer 3. On top of this is a 2 inch (5 cm) standard, open cell viscoelastic (visco) layer 2. The top layer 1 is a 2 inch (5 cm) layer of AW Foam. It will be appreciated that the dimensions given in the examples and descriptions of the various Figures are merely illustrative and are not intended to be limiting. Throughout the drawings, the same or similar reference numerals will be used for the same or similar structures.

FIG. 3 is the second example construction using a cushion and/or mattress application. The base of the section is a prime foam layer 3. On top of this is a 2 inch (5 cm) layer of AW Foam 1 followed by a 2 inch (5 cm) layer 2 of standard, open cell viscoelastic foam.

FIG. 4 is the third example construction using a cushion and/or mattress application. The base of the section is a prime foam layer 3. On top of this is a 2 inch (5 cm) layer of AW Foam 1 followed by a 0.75 inch (1.9 cm) layer 3 of prime foam. The top layer is a second 2 inch (5 cm) layer of AW Foam 1.

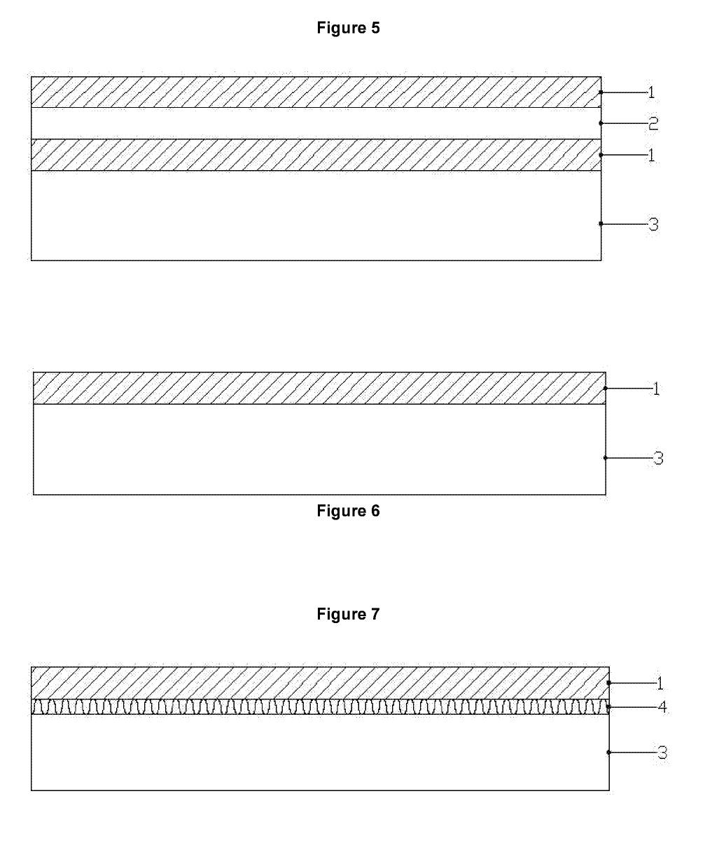

FIG. 5 is the fourth example construction using a cushion and/or mattress application. The base of the section is a prime foam layer 3. On top of this is a 2 inch (5 cm) layer of AW Foam 1 followed by a 2 inch (5 cm) layer 2 of standard, open cell viscoelastic foam. The top layer is a second 2 inch (5 cm) layer of AW Foam 1.

FIG. 6 is the fifth example construction using a cushion and/or mattress application. The base of the section is a prime foam layer 3. On top of this is a 3 inch layer of AW Foam 1.

FIG. 7 is the sixth example construction using a cushion and/or mattress application. The base of the section is a prime foam layer 3. On top of this is a 3 inch (7.6 cm) layer of AW Foam 1. The interface 4 between the two layers is a non-planar convolution, which may be made by convoluting the surface of either or both interfacing layers.

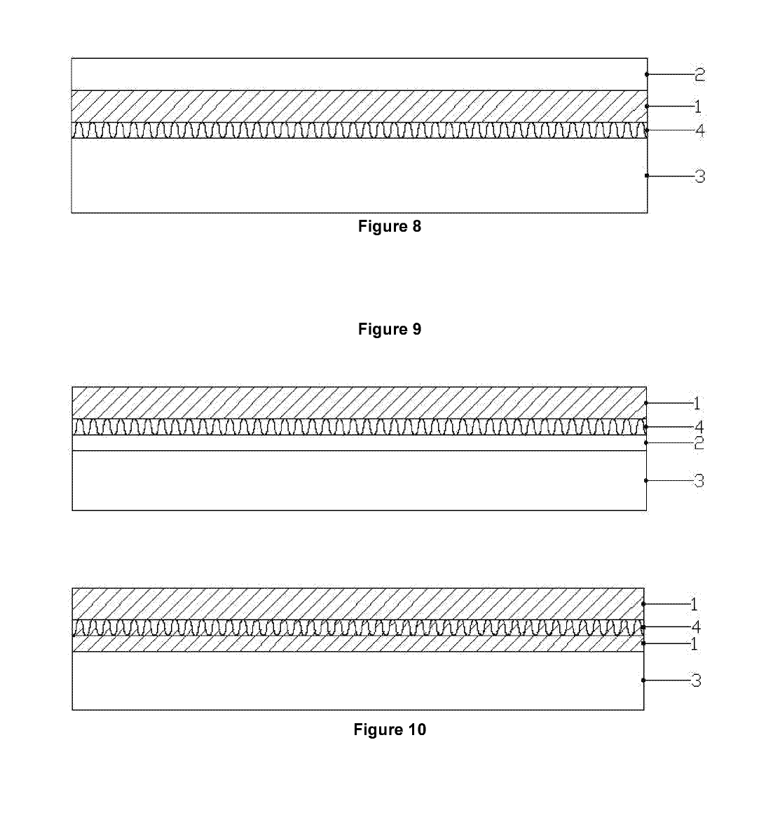

FIG. 8 is the seventh example construction using a cushion and/or mattress application. The base of the section is a prime foam layer 3. On top of this is a 2 inch (5 cm) layer of AW Foam 1. The interface 4 between the two layers is a non-planar convolution, which may be made by convoluting the surface of either or both interfacing layers. The top of this example is a 2 inch (5 cm) layer 2 of standard, open-cell viscoelastic foam.

FIG. 9 is the eighth example construction using a cushion and/or mattress application. The base of the section is a prime foam layer 3. Above this is a 2 inch (5 cm) layer 2 of standard, open-cell viscoelastic foam. On top of this is a 2 inch layer (5 cm) of AW Foam 1. The interface 4 between the two layers is a non-planar convolution, which may be made by convoluting the surface of either or both interfacing layers.

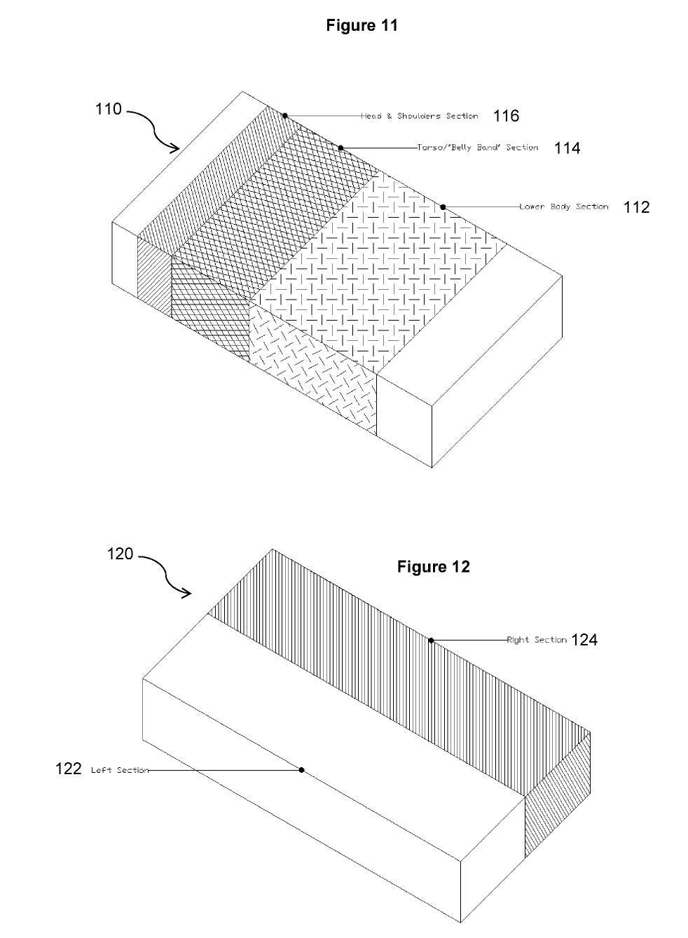

FIG. 10 is the ninth example construction using a cushion and/or mattress application. The base of the section is a prime foam layer 3. Above this is a 2 inch (5 cm) layer of AW Foam 1. On top of this is another 2 inch (5 cm) layer of AW Foam 1. The interface 4 between the two layers is a non-planar convolution, which may be made by convoluting the surface of either or both interfacing layers.

FIG. 11 is an example breakdown of lateral mattress zones or sections in a mattress 110. These zones include: lower body zone or section 112, torso/“belly band” zone or section 114, and head and shoulders zone or section 116. These zones or sections may or may not include AW Foams, example constructions, other mattress layer constructions, or any variation thereof. Furthermore, the zones shown are not limiting, but used as an example to show the possibility of utilizing enhanced thermally dissipating layers in specific areas of cushions and/or a mattress.

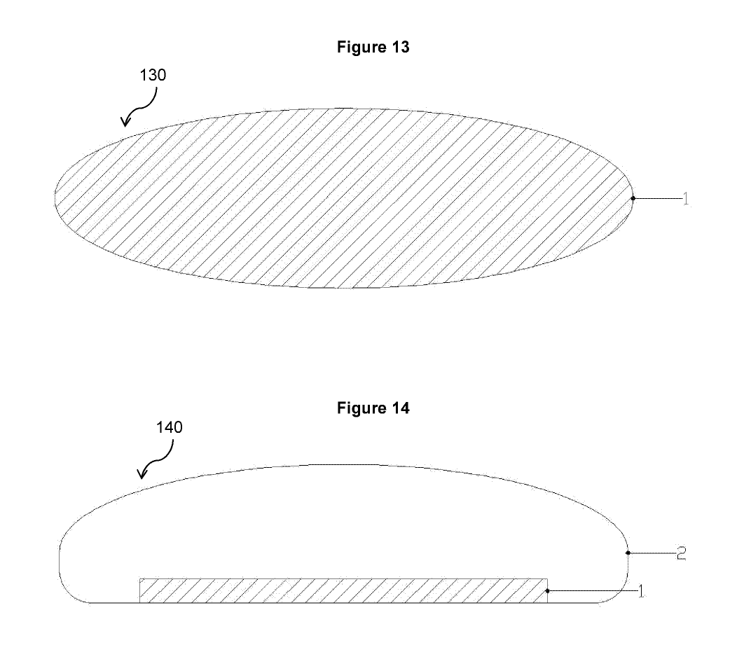

FIG. 12 is an example breakdown of longitudinal mattress zones 122 and 124 in a mattress 120. These zones include left section 122 and right section 124. These zones or sections 122 and 124 may or may not include AW Foams, example constructions, other mattress layer constructions, or any variation thereof. Furthermore, the zones shown are not limiting, but used as an example to show the possibility of utilizing enhanced thermally dissipating layers in specific areas of cushions and/or a mattress.

FIGS. 13 and 14 are depictions of molded pillow systems. FIG. 13 is a pillow 130 molded entirely out of AW Foam 1. FIG. 14 shows a pillow 140 using AW Foam 1 as a region within the overall pillow structure 2.

The invention will now be described more specifically with respect to particular formulations, methods and compositions herein to further illustrate the invention, but which examples are not intended to limit the methods and compositions herein in any way.

### EXAMPLE 1

### Control Foam

A two component system was obtained from Peterson Chemical Technology. The PCT-L4930B system consisted of a “B” side containing polyols, surfactants, blowing and gelation catalysts and water, and the “A” side (PCT-M142A) consisted of an isocyanate compound. In a 32 oz (0.95 L) mix cup, 103.0 parts of the PCT-L4930B “B” side was added. The components were mixed for approximately 45 seconds before adding 45.11 parts of PCT-M142A “A” side component, mixed an additional 10 seconds and poured into a 9″×9″ (23 cm×23 cm) cake box and allowed to rise and cure in a room temperature environment. A flexible polyurethane foam was produced which is the control foam labeled Example 1 in table 1. Physical properties such as density, IFD, and airflow were measured.

### EXAMPLE 2

A two component system was obtained from Peterson Chemical Technology. The PCT-L6163B system consisted of a “B” side containing polyols, surfactants, blowing and gelation catalysts and water, and the “A” side (PCT-M142A) consisted of an isocyanate compound. In a 32 oz (0.95 L) mix cup, the components were added as follows: 102.9 parts of the PCT-L6163B “B” side and 8.5 parts of aromatic red cedar particles with particle sizes less 1000 microns. The red cedar particles were obtained by cross-cutting on a table saw, collecting the table saw cutting, and sifting through a 1000 micron screen. The components were mixed for approximately 45 seconds before adding 44.06 parts of PCT-M142A “A” side component, mixed an additional 10 seconds and poured into a 9″×9″ (23 cm×23 cm) cake box and allowed to rise and cure in a room temperature environment. A flexible polyurethane foam was produced which is labeled Example 2 in table 1. Physical properties such as density, IFD, and airflow were measured.

### EXAMPLE 3

A two component system was obtained from Peterson Chemical Technology. The PCT-L6153B system consisted of a “B” side containing polyols, surfactants, blowing and gelation catalysts and water, and the “A” side (PCT-M142A) consisted of an isocyanate compound. In a 32 oz (0.95 L) mix cup, the components were added as follows: 102.9 parts of the PCT-L6153B “B” side, 8.5 parts of aromatic red cedar particles with particle sizes less 1000 microns, and 0.03 parts of cedarwood essential oil. The red cedar particles were obtained by cross-cutting on a table saw, collecting the table saw cutting, and sifting through a 1000 micron screen. The red cedar particles were soaked with the cedarwood essential oil prior to adding to “B” side mixture. The components were mixed for approximately 45 seconds before adding 44.06 parts of PCT-M142A “A” side component, mixed an additional 10 seconds and poured into a 9″×9″ (23 cm×23 cm) cake box and allowed to rise and cure in a room temperature environment. A flexible polyurethane foam was produced which is the labeled Example 3 in table 1. Physical properties such as density, IFD, and airflow were measured.

### Discussion of Results

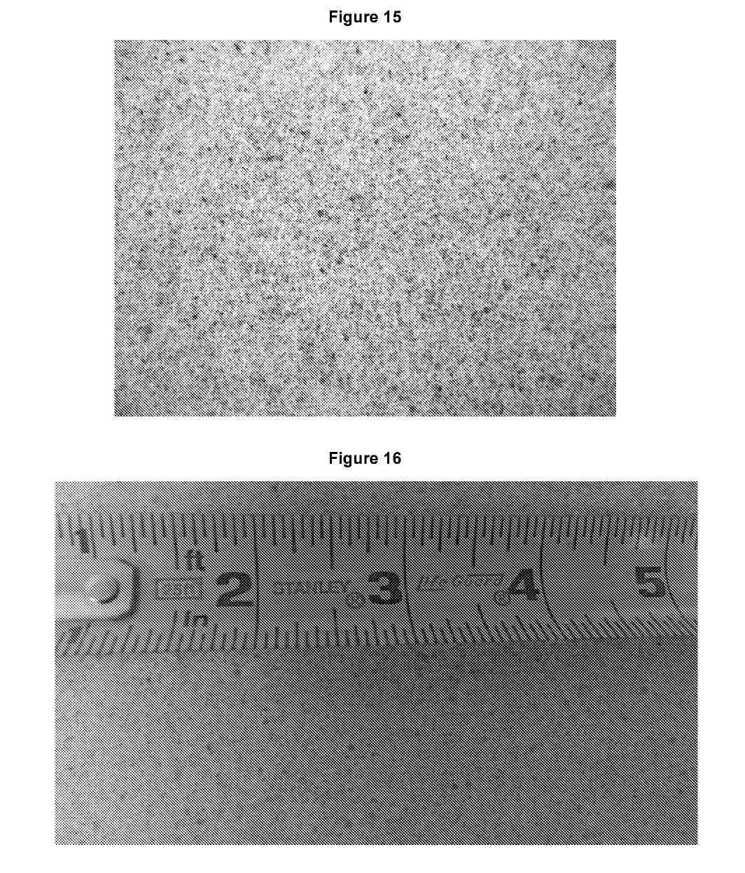

Table 1 shows the formula and test results for the foams produced by following the procedures of Examples 1, 2, and 3. FIG. 15 is a photograph of aromatic wood particles in open cell flexible polyurethane foam from Example 2. FIG. 16 is a close-up photograph of a viscoelastic polyurethane foam made with 10 wt % cedar powder added showing the aromatic wood particles to scale next to a ruler.

TABLE 1
Comparison of Formula and Properties of Control
(Example 1) and AW Foam Examples 2 and 3

| | | Ex. 1 | | |
|---|---|---|---|---|
| Sample | Units | (Control) | Ex. 2 | Ex. 3 |
| PCT-L4980B | pph¹ | 103.0 | | |
| PCT-L6163B | pph | | 102.9 | |
| PCT-L6153B | pph | | | 102.9 |
| Red Aromatic | pph | | 8.5 | 8.5 |
| Cedar Particles | | | | |
| Cedarwood | pph | | | 0.03 |
| Essential Oil | | | | |
| PCT-M142A | pph | 45.11 | 44.06 | 44.06 |
| Cream | sec | 24 | 27 | 29 |
| Rise | sec | 140 | 148 | 140 |
| Settle | in (cm) | 0 (0) | 0 (0) | 0 (0) |
| Density | pcf | 3.50 (56.1) | 3.54 (56.7) | 3.58 (57.3) |
| | (kg/m³) | | | |
| Airflow² | SCFM | 3.47 (5.9) | 3.77 (6.4) | 3.07 (5.20) |
| | (m³/hr) | | | |
| 25% IFD³ | lb_{f}/50 in² | 13.3 | 10.7 | 12.3 |
| Appearance | | Uniform | Uniform | Uniform |
| | | open cell | open cell | open cell |
| | | foam | foam with | foam with |
| | | | randomly | randomly |
| | | | distributed | distributed |
| | | | particles | particles |
| Odor | | Low odor - | Mild cedar | Strong |
| | | typical | fragrance | cedar |
| | | foam | | fragrance |

¹pph = parts per hundred
²Airflow: ASTM D 3574 G
³25% IFD: ASTM D 3574 B

Many modifications may be made in the methods of and implementation of this invention without departing from the scope thereof that are defined only in the appended claims. For instance, various combinations of polyols, isocyanates, catalysts, wood particulates, aromatic wood particulates, aromatic essential oils and fragrances, and other additives, and processing pressures and conditions besides those explicitly mentioned herein are expected to be useful.

The words “comprising” and “comprises” as used throughout the claims are to be interpreted as “including but not limited to”. The present invention may suitably comprise, consist or consist essentially of the elements disclosed and may be practiced in the absence of an element not disclosed. In a non-limiting instance, there may be provided AW Foam that consists essentially of or consists of a flexible cellular foam and aromatic wood material particulates dispersed in the flexible cellular foam.

Alternatively, an aromatic wood-containing latex foam may consist essentially of or consists of cross-linked latex foam and aromatic wood material particulates dispersed in the cross-linked latex foam. There may also be provided aromatic wood-containing melamine foam consisting essentially of or consisting of cross-linked melamine foam and aromatic wood material particulates dispersed in the cross-linked melamine foam.

1-24. (canceled)

25. An aromatic wood foam (AW Foam) comprising:
a flexible cellular foam, and
aromatic wood particulates dispersed in the flexible cellular foam.

26. The AW Foam of claim 25 where the aromatic wood particulates are aromatic by their nature.

27. The AW Foam of claim 25 where the aromatic wood particulates contain at least one essential oil or fragrance, where the aromatic wood particulates are produced by a process selected from the group consisting of infusing, coating, penetrating, and combinations thereof with the at least one essential oil or fragrance.

28. The AW Foam of claim 25 having an aromatic fragrance, where the aromatic fragrance of the wood particulates in a bedding or cushioning foam is present in an amount effective to have insect repellant properties.

29. The AW Foam of claim 25 where the aromatic wood particulates comprise wood particles selected from the group consisting of Spanish Cedar, Eastern Red Cedar, Juniper, Aromatic Red Cedar, White Cedar, Eucalyptus, Cypress, Western Red Cedar, and combinations thereof.

30. The AW Foam of claim 25 where the AW Foam is produced by a method comprising:
introducing aromatic wood particulates (AW particulates) into a mixture of flexible cellular foam-forming components comprising a polyol and an isocyanate; and
polymerizing the polyol and the isocyanate to form the flexible cellular foam.

31. An article of manufacture comprising the AW Foam of claim 25 where the article of manufacture is selected from the group consisting of medical cushioning foams, mattresses, pillows, bedding products, mattress pillow toppers, quilted mattress toppers, mattress toppers, pet beds, indoor cushioning foams, outdoor cushioning foams, outdoor bedding pads, outdoor pillows, and combinations thereof.

32. The AW Foam of claim 25 where the flexible cellular foam is selected from the group consisting of an open cell polyurethane foam, partially-open cell polyurethane foam, open cell polyester polyurethane foam, partially-open cell polyester polyurethane foam, and combinations thereof.

33. The AW Foam of claim 27 wherein the essential oil or fragrance is selected from the group consisting cedarwood oil, citronella oil, cypress oil, eucalyptus oil, juniper berry oil, lavender oil, lemon oil, lemongrass oil, lime oil, mint oil, peppermint oil, pine oil, red cedar oil, western red cedar oil, wintergreen oil, and combinations thereof.

34. The AW Foam of claim 25 wherein the AW particulates have a form selected from the group consisting of shavings, splinters, flakes, powders, spherical, and combinations thereof.

35. The AW Foam of claim 25 wherein the AW particulates have an average particle size of less than about 3000 microns.

36. The AW Foam of claim 25 wherein the AW particulates are present in the range of from about 0.1 to about 50% by weight in the AW Foam.

37. The AW Foam of claim 25 wherein the AW Foam comprises a structure selected from the group consisting of a solid sheet, a perforated sheet, a non-planar sheet, a planar sheet, a textured sheet, and combinations thereof.

38. The AW Foam of claim 25 wherein the AW Foam is adhered to a layering substrate.

39. A cushion foam comprising a layer or layers comprised of the AW Foam of claim 25.

40. A mattress comprising at least one layer comprised of the AW Foam of claim 25.

41. A mattress topper pad comprising at least one layer comprised of the AW Foam of claim 25.

42. A pet bed comprising at least one layer comprised of the AW Foam of claim 25.

43. A pillow comprising the AW Foam of claim 25.

44. An article of manufacture selected from the group consisting of a cushion foam, a mattress, a mattress topper pad, and combinations thereof, where the article of manufacture comprises at least one zone selected from the group consisting of a longitudinal zone, a lateral zone, and combinations thereof, where the at least one zone comprises the AW Foam of claim 25.

45. An article of manufacture selected from the group consisting of medical cushioning foams, mattresses, pillows, bedding products, mattress pillow toppers, quilted mattress toppers, mattress toppers, pet beds, indoor cushioning foams, outdoor cushioning foams, outdoor bedding pads, outdoor pillows, and combinations thereof, where the article of manufacture further comprises at least one layer comprising the AW Foam of claim 25.

46. An aromatic wood (AW) latex foam comprising:
a cross-linked latex foam; and
aromatic wood particulates dispersed in the cross-linked latex foam.

47. An aromatic wood (AW) melamine foam comprising:
a cross-linked melamine foam; and
aromatic wood particulates dispersed in the cross-linked melamine foam.
